(19) United States
(12) Patent Application Publication
(10) Pub. No.: US 2010/0003200 A1
(43) Pub. Date: Jan. 7, 2010

(54) AEROSOL DEVICE AND HAIR STYLING METHOD

(75) Inventors: Claude Dubief, Le Chesnay (FR); Franck Giroud, Clichy (FR); Isabelle Rollat-Corvol, Paris (FR)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'OREAL, Paris (FR)

(21) Appl. No.: 12/562,436

(22) Filed: Sep. 18, 2009

Related U.S. Application Data
(63) Continuation of application No. 10/448,186, filed on May 29, 2003.
(60) Provisional application No. 60/387,423, filed on Jun. 11, 2002.

(30) Foreign Application Priority Data
May 31, 2002 (FR) 0206737

(51) Int. Cl. A61K 8/90; A61Q 5/06

(52) U.S. Cl. 424/47

(57) ABSTRACT

An aerosol device has a first compartment containing a styling composition and a second compartment containing a compressed gas. The styling composition consists essentially of water, at least one linear block copolymer having at least one hydrophilic block and at least one hydrophobic block, the hydrophilic block representing at least 30% by weight of the linear block copolymer, and one or more additives. The styling composition excludes block copolymers of ethylene oxide and of propylene oxide, block copolymers containing urethane units, block copolymers containing siloxane units, and volatile organic compounds.

### CROSS-REFERENCE TO RELATED APPLICATIONS

This application is a continuation of U.S. application Ser. No. 10/448,186, filed on May 29, 2003, the disclosure of which is hereby incorporated by reference herein, which claims priority from French Application No. 0206737 filed on May 31, 2002 and the benefit of the filing date of U.S. Provisional Application No. 60/387,423 filed on Jun. 11, 2002.

### BACKGROUND OF THE INVENTION

The present invention relates to an aerosol device comprising a styling composition containing at least one amphiphilic block polymer and a compressed gas and to a styling process.

Styling compositions such as lacquers and sprays, packaged in aerosol spray form, are generally composed of a liquid phase comprising at least one film-forming polymer in an alcoholic or aqueous-alcoholic cosmetically acceptable medium, and a propellant, which is a liquefied gas under reduced pressure or dissolved in the liquid phase. In the field of hair products, it is desired to manufacture aerosol lacquers comprising no volatile organic compound such as ethanol or dimethyl ether, for essentially ecological reasons, while at the same time maintaining good hairstyle shaping and hold properties.

U.S. Pat. No. 5,626,840 describes hair fixing compositions that may be in aerosol form. They comprise a polyurethane in an aqueous or aqueous-alcoholic medium, a mineral or organic base and a solvent which may be water or a mixture of water and a polar organic solvent. The composition described in U.S. Pat. No. 5,626,840 is said to contain a marked decrease in volatile organic compounds in the styling compositions.

### SUMMARY OF THE INVENTION

Applicants have surprisingly discovered that by using a device comprising a first compartment containing a styling composition including at least one linear block copolymer, and a compressed gas in a second compartment, it is possible to obtain a lacquer that has shaping and hold properties that are at the very least equivalent to prior art lacquers that contain volatile organic compounds. According to one or more embodiments, the linear block copolymer includes at least one hydrophilic block and at least one hydrophobic block, the hydrophilic block(s) representing at least about 30% by weight of the linear block copolymer, excluding block copolymers of ethylene oxide and of propylene oxide, block copolymers containing urethane units and block copolymers containing siloxane units.

One embodiment of the present invention relates to a two-compartment aerosol device comprising a first compartment containing a styling composition including at least one linear block copolymer including at least one hydrophilic block and at least one hydrophobic block, the hydrophilic block(s) representing at least about 30% by weight of the linear block copolymer, excluding block copolymers of ethylene oxide and of propylene oxide, block copolymers containing urethane units and block copolymers containing siloxane units, and a second compartment containing a compressed gas.

Another embodiment of the present invention relates to a styling process using the device of the invention. Other characteristics, aspects and advantages of the invention will be apparent on reading the description and the various examples that follow.

Preferably, the compressed gas is chosen from air, nitrogen and carbon dioxide, and mixtures thereof, air being particularly preferred. The compressed gas is preferably used at a pressure of between 1 and 12 bar and more preferably between 9 and 11 bar. The linear block copolymers that may be used according to one or more embodiments of the present invention are “amphiphilic” copolymers, i.e. copolymers comprising both hydrophobic blocks and hydrophilic blocks.

### DETAILED DESCRIPTION

Before describing several exemplary embodiments of the invention, it is to be understood that the invention is not limited to the details of construction or process steps set forth in the following description. The invention is capable of other embodiments and of being practiced or carried out in various ways.

This, according to one embodiment, the invention comprises an aerosol device comprising a first compartment containing a styling composition including at least one linear block copolymer including at least one hydrophilic block and at least one hydrophobic block, said hydrophilic block representing at least about 30% by weight of said linear block copolymer, said styling composition excluding block copolymers of ethylene oxide and of propylene oxide, block copolymers containing urethane units and block copolymers containing siloxane units, and a second compartment containing a compressed gas.

The compressed gas may be chosen from air, nitrogen and carbon dioxide, and mixtures thereof, air being particularly preferred. The compressed gas is preferably used at a pressure of between 1 and 12 bar and more preferably between 9 and 11 bar. The linear block copolymers that may be used according to one or more embodiments of the present invention are “amphiphilic” copolymers, i.e. copolymers comprising both hydrophobic blocks and hydrophilic blocks.

According to one or more embodiments of the present invention, the term “hydrophobic blocks” means blocks comprising at least about 75 mol % of water-insoluble monomers, and the term “hydrophilic blocks” means blocks comprising at least about 75 mol % of water-soluble monomers.

The water-soluble monomers forming the hydrophilic blocks of the block copolymers used in one or more embodiments of the present invention may be of anionic, nonionic or cationic nature and may be used alone or in the form of a mixture containing two or more different monomers. Examples of anionic water-soluble monomers include, but are not limited to, ethylenically unsaturated carboxylic acids, such as acrylic acid, methacrylic acid, itaconic acid, fumaric acid, crotonic acid, maleic acid, 2-acrylamido-2-methylpropanesulphonic acid, styrenesulphonic acid, vinylsulphonic acid and vinylphosphonic acid.

The nonionic water-soluble monomers include, but are not limited to, acrylamide, C₁₋₆ N-alkyl or C₁₋₃ N, N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol (copolymerized in the form of vinyl acetate and then hydrolysed), ethylene oxide, hydroxyethyl acrylate, hydroxypropyl acrylate, hydroxyethyl methacrylate and hydroxypropyl methacrylate.

Finally, the cationic water-soluble monomers include, for example, dimethyldiallylammonium chloride, methylvinylimidazolium chloride, 2-vinylpyridine, 4-vinylpyridine, 2-methyl-5-vinylpyridine, vinylamine, the monomers of formula: H₂C═CR₁—CO—X₂, in which R₁ represents a hydrogen atom or a methyl group, X₂ represents a linear or branched C₁₋₆ hydrocarbon-based group bearing at least one primary, secondary or tertiary amine function or at least one quaternary nitrogen atom, or a group of formula NHR₂ or of formula NR₂R₃ in which R₂ and R₃ represent, independently of each other, a linear or branched C₁₋₆ hydrocarbon-based group bearing at least one primary, secondary or tertiary amine function or at least one quaternary nitrogen atom.

The water-insoluble monomers forming the hydrophobic blocks of the block copolymers are preferably chosen from vinylaromatic monomers such as styrene and its alkyl derivatives, for instance 4-butylstyrene, α-methylstyrene and vinyltoluene, dienes such as butadiene and 1,3-hexadiene, and alkyl derivatives of dienes such as isoprene and dimethylbutadiene, chloroprene, C₁₋₁₀ alkyl, C₆₋₁₀ aryl or C₆₋₁₀ aralkyl acrylates and C₁₋₁₀ alkyl, C₆₋₁₀ aryl, or C₆₋₁₀ aralkyl methacrylates, for instance methyl, ethyl, n-butyl, 2-ethylhexyl, tert-butyl, isobornyl, phenyl or benzyl (meth)acrylates, vinyl acetate, vinyl ethers of formula CH₂═CH—O—R and allyl ethers of formula CH₂═CH—CH₂—O—R in which R represents a C₁₋₆ alkyl group, acrylonitrile, vinyl chloride, vinylidene chloride, caprolactone, ethylene, propylene, vinyl monomers that are fluorinated or that contain a perfluoro chain, such as fluoroalkyl acrylates and methacrylates or alkyl fluoroacrylates.

As indicated above, with respect to the definition of the hydrophobic and hydrophilic blocks of the block copolymers, the water-insoluble monomers and the water-soluble monomers represent at least about 75 mol %, respectively, of the hydrophobic and hydrophilic blocks. In other words, each hydrophobic block may comprise up to about 25 mol % of one or more water-soluble monomers. This proportion is preferably not more than about 10 mol % and more preferably less than or equal to about 5 mol %. Similarly, each hydrophilic block may comprise up to about 25 mol %, preferably up to about 10 mol % and more preferably up to about 5 mol % of one or more water-insoluble monomers.

The linear block copolymers used according to one or more embodiments also obviously include those in which the hydrophilic blocks and the hydrophobic blocks consist exclusively of water-soluble monomers and of water-insoluble monomers, respectively. These blocks may be homopolymer blocks or copolymer blocks containing two or more than two different monomers of the same type. Preferably, the linear block copolymers of the invention are water-soluble or water-dispersible, and even more preferably they are water-soluble.

The term “water-soluble monomer or polymer” means a monomer or polymer which, when introduced into water at a temperature of about 25° C. and at a weight concentration equal to about 0.5%, produces a macroscopically homogeneous and transparent solution, i.e. a solution with a light transmittance value, at a wavelength equal to about 500 nm, through a sample about 1 cm thick, of at least about 70% and preferably of at least about 80%. The water-dispersible copolymers according to the invention may be in the form of lattices or pseudolattices.

Preferably, the linear block copolymers according to one or more embodiments of the invention are water-soluble. This ability to be dissolved or finely dispersed in water is linked to the large proportion of hydrophilic blocks in the amphiphilic block copolymers.

This proportion is preferably at least about 30% by weight and is more preferably greater than or equal to about 60% by weight, without, however, exceeding about 97% by weight and preferably about 95% by weight.

The number-average molecular mass of each block, whether it is hydrophobic or hydrophilic, a copolymer or a homopolymer, is preferably between about 500 and about 100 000 and in particular between about 500 and about 50 000, with a polydispersity index (M_{w}/Mₙ) of between about 1.01 and about 3.0 and preferably between about 1.1 and about 2.5.

The linear block copolymers used in the present invention may be:
diblock copolymers of formula AB,
triblock copolymers of formula ABA or BAB, and
multiblock copolymers comprising at least two hydrophilic blocks and at least two hydrophobic blocks arranged alternately, each A representing a hydrophilic block and each B representing a hydrophobic block, the blocks A of the same polymer possibly being identical or different and the blocks B of the same polymer possibly being identical or different.

Diblock copolymers and triblock copolymers comprising a hydrophilic central block and two hydrophobic side blocks are particularly preferred. The block polymers of the invention may be prepared by the synthetic processes conventionally used for obtaining block polymers. Mention may be made, for example, of anionic or cationic polymerization and controlled free-radical polymerization (see “New Method of Polymer Synthesis”, Blackie Academic & Professional, London, 1995, Volume 2, page 1, or Trends Polym. Sci. 4, page 183 (1996) by C. J. Hawker), which may be used according to different processes, for instance atom transfer radical polymerization (ATRP) (see JACS, 117, page 5614 (1995) from Matyjasezwski et al.), and the method using radicals such as nitroxides (Georges et al., Macromolecules, 1993, 26, 2987). These processes may also be used to obtain just one of the two types of blocks of the polymer of the invention, the other block being introduced into the final polymer by means of the initiator used or alternatively via a coupling reaction between the hydrophilic and hydrophobic blocks.

The amount of amphiphilic linear block copolymers in the compositions of the present invention depends on a large number of parameters, among which mention may be made of the molecular mass of the copolymers, the number and size of the hydrophilic and hydrophobic blocks, the amount of polymers that are beneficial to the hair, and especially the viscosity of the composition that it is desired to obtain. Satisfactory results are generally obtained with an amount of linear block copolymers of between about 0.01% and about 10% by weight and preferably between about 0.1% and about 5% by weight relative to the weight of the hair composition.

The styling composition contained in the device according to one or more embodiments of the invention advantageously has a water content of between about 30% and about 99.9% by weight, preferably between about 50% and about 99% by weight and even more preferably between about 75% and about 98% by weight relative to the total weight of the said composition.

The styling composition may also comprise additives such as silicones in soluble, dispersed or microdispersed form, treating active agents, moisturizers, for instance glycerol, UV-screening agents, acids, bases, plasticizers, solubilizers, preserving agents, vitamins and provitamins, colorants, pigments, anionic, cationic, nonionic or amphoteric surfactants, fragrances and anti-corrosion agents, and mixtures thereof. A person skilled in the art will take care to select the optional additives and the amount thereof such that they do not harm the properties of the compositions of the present invention. These additives are especially present in the composition according to the invention in an amount ranging from about 0 to about 20% by weight relative to the total weight of the composition.

Preferably, the two-compartment aerosol device consists of an outer aerosol can comprising an inner pocket hermetically welded to a valve. The composition is introduced into the inner pocket and a compressed gas is introduced between the pocket and the can at a pressure that is sufficient to eject the product in the form of a spray through the orifice of a nozzle. Such a device is sold under the name EP Spray by the company EP Spray System SA. The aerosol devices of the invention preferably contain hair lacquers.

The present invention also relates to a styling process which consists in vaporizing the styling composition contained in the aerosol device according to one or more previously described embodiments of the invention onto wet or dry hair.

The examples that follow are given as illustrations of the present invention.

### Example 1

A styling composition was prepared using the ingredients below:

| | % by weight |
|---|---|
| ⁽¹⁾ Cationic block copolymer formed from a | 5 AM* |
| polystyrene block and a poly(N-methyl-N- | |
| vinylpyridinium iodide) block | |
| Demineralized water qs | 100 |

*Active material

The composition prepared above was introduced into an aerosol distribution device sold under the name EP Spray by the company EP Spray System S.A. described above. A valve of reference 6001 format D6 is attached to a standard aerosol can and the diffuser is a vortex-nozzle diffuser.

The pocket is filled with the composition as indicated above. Compressed air is introduced between the pocket and the can. The composition was vaporized onto dry hair. The spraying takes place in the form of a gentle spray. After drying, a head of hair with good hold is obtained.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims and their equivalents.

1. An aerosol device, comprising:
a first compartment containing a styling composition; and
a second compartment containing a compressed gas which is not a volatile organic compound;
the styling composition consisting essentially of (a) water, (b) at least one linear block copolymer having at least one hydrophilic block and at least one hydrophobic block, the hydrophilic block representing at least about 30% by weight of the linear block copolymer, and (c) one or more additives;
the styling composition excluding block copolymers of ethylene oxide and of propylene oxide, block copolymers containing urethane units, block copolymers containing siloxane units, and volatile organic compounds; and
the additives being selected from the group consisting of silicones in soluble, dispersed or microdispersed form, treating active agents, moisturizers, UV-screening agents, acids, bases, plasticizers, solubilizers, preserving agents, vitamins and provitamins, colorants, pigments, anionic, cationic, nonionic or amphoteric surfactants, fragrances, anti-corrosion agents, and mixtures thereof.

2. An aerosol device according to claim 1, wherein the compressed gas is selected from the group consisting of air, nitrogen, carbon dioxide, and mixtures thereof.

3. An aerosol device according to claim 2, wherein the pressure of the compressed gas is between 1 and 12 bar.

4. An aerosol device according to claim 3, wherein the pressure of the compressed gas is between 9 and 11 bar.

5. An aerosol device according to claim 1, wherein the linear block copolymer is selected from the group consisting of diblock copolymers of formula AB, triblock copolymers of formula ABA or BAB and multiblock copolymers comprising at least two hydrophilic blocks and at least two hydrophobic blocks arranged alternately, each A representing a hydrophilic block and each B representing a hydrophobic block, the blocks A of the same polymer being identical or different and the blocks B of the same polymer being identical or different.

6. An aerosol device according to claim 5, wherein the linear block copolymer is selected from the group consisting of diblock copolymers and triblock copolymers including a hydrophilic central block and two hydrophobic side blocks.

7. An aerosol device according to claim 1, wherein the hydrophilic block is formed from water-soluble monomers selected from the group consisting of anionic water-soluble monomers, nonionic water-soluble monomers and cationic water-soluble monomers and mixtures thereof.

8. An aerosol device according to claim 7, wherein the anionic water-soluble monomers are selected from the group consisting of ethylenically unsaturated carboxylic acids, 2-acrylamido-2-methylpropanesulphonic acid, styrenesulphonic acid, vinylsulphonic acid and vinylphosphonic acid.

9. An aerosol device according to claim 7, wherein the nonionic water-soluble monomers are selected from the group consisting of acrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol, ethylene oxide, hydroxyethyl acrylate, hydroxypropyl acrylate, hydroxyethyl methacrylate and hydroxypropyl methacrylate.

10. An aerosol device according to claim 7, wherein the cationic water-soluble monomers are selected from the group consisting of dimethyldiallylammonium chloride, methylvinylimidazolium chloride, 2-vinylpyridine, 4-vinylpyridine, 2-methyl-5-vinylpyridine, vinylamine, the monomers of formula H₂C═CR₁—CO—X₂ in which R₁ represents a hydrogen atom or a methyl group, X₂ represents a linear or branched C₁₋₆ hydrocarbon-based group bearing at least one primary, secondary or tertiary amine function or at least one quaternary nitrogen atom, or a group of formula NHR₂ or of formula NR₂R₃ in which R₂ and R₃ represent, independently of each other, a linear or branched C₁₋₆ hydrocarbon-based group bearing at least one primary, secondary or tertiary amine function or at least one quaternary nitrogen atom.

11. An aerosol device according claim 7, wherein the hydrophilic block contains up to about 25 mol % of one or more water-insoluble monomers selected from the group consisting of vinylaromatic monomers, dienes and alkyl derivatives of dienes, chloroprene, C₁₋₁₀ alkyl, C₆₋₁₀ aryl or C₁₋₁₀ aralkyl acrylates, C₁₋₁₀ alkyl, C₆₋₁₀ aryl or C₁₋₁₀ aralkyl methacrylates, vinyl acetate, vinyl ethers of formula CH₂═CH—O—R and allyl ethers of formula CH₂═CH—CH₂—O—R in which R represents a C₁₋₆ alkyl group, acrylonitrile, vinyl chloride, vinylidene chloride, caprolactone, ethylene, propylene, vinyl monomers that are fluorinated or that contain a perfluoro chain, and combinations thereof.

12. An aerosol device according claim 7, wherein the hydrophilic block contains up to about 10 mol % of one or more water-insoluble monomers selected from the group consisting of vinylaromatic monomers, dienes and alkyl derivatives of dienes, chloroprene, C₁₋₁₀ alkyl, C₆₋₁₀ aryl or C₁₋₁₀ aralkyl acrylates, C₁₋₁₀ alkyl, C₆₋₁₀ aryl or C₁₋₁₀ aralkyl methacrylates, vinyl acetate, vinyl ethers of formula CH₂═CH—O—R and allyl ethers of formula CH₂═CH—CH₂—O—R in which R represents a C₁₋₆ alkyl group, acrylonitrile, vinyl chloride, vinylidene chloride, caprolactone, ethylene, propylene, vinyl monomers that are fluorinated or that contain a perfluoro chain, and combinations thereof.

13. An aerosol device according claim 7, wherein the hydrophilic block contains up to about 5 mol % of one or more water-insoluble monomers selected from the group consisting of vinylaromatic monomers, dienes and alkyl derivatives of dienes, chloroprene, C₁₋₁₀ alkyl, C₆₋₁₀ aryl or C₁₋₁₀ aralkyl acrylates, C₁₋₁₀ alkyl, C₆₋₁₀ aryl or C₁₋₁₀ aralkyl methacrylates, vinyl acetate, vinyl ethers of formula CH₂═CH—O—R and allyl ethers of formula CH₂═CH—CH₂—O—R in which R represents a C₁₋₆ alkyl group, acrylonitrile, vinyl chloride, vinylidene chloride, caprolactone, ethylene, propylene, vinyl monomers that are fluorinated or that contain a perfluoro chain, and combinations thereof.

14. An aerosol device according to claim 1, wherein the hydrophobic block is formed from water-insoluble monomers selected from the group consisting of vinylaromatic monomers, dienes and alkyl derivatives of dienes, chloroprene, C₁₋₁₀ alkyl, C₆₋₁₀ aryl or C₁₋₁₀ aralkyl acrylates, C₁₋₁₀ alkyl, C₆₋₁₀ aryl or C₁₋₁₀ aralkyl methacrylates, vinyl acetate, vinyl ethers of formula CH₂═CH—O—R and allyl ethers of formula CH₂═CH—CH₂—O—R in which R represents a C₁₋₆ alkyl group, acrylonitrile, vinyl chloride, vinylidene chloride, caprolactone, ethylene, propylene, vinyl monomers that are fluorinated or that contain a perfluoro chain, and combinations thereof.

15. An aerosol device according to claim 14, wherein the hydrophobic block contains up to about 25 mol % of one or more water-soluble monomers selected from the group consisting of ethylenically unsaturated carboxylic acids, 2-acrylamido-2-methylpropanesulphonic acid, styrenesulphonic acid, vinylsulphonic acid, vinylphosphonic acid, acrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol, ethylene oxide, hydroxyethyl acrylate, hydroxypropyl acrylate, hydroxyethyl methacrylate, hydroxypropyl methacrylate, and dimethyldiallylammonium chloride, methylvinylimidazolium chloride, 2-vinylpyridine, 4-vinylpyridine, 2-methyl-5-vinylpyridine, vinylamine, the monomers of formula H₂C═CR₁—CO—X₂ in which R₁ represents a hydrogen atom or a methyl group, X₂ represents a linear or branched C₁₋₆ hydrocarbon-based group bearing at least one primary, secondary or tertiary amine function or at least one quaternary nitrogen atom, or a group of formula NHR₂ or of formula NR₂R₃ in which R₂ and R₃ represent, independently of each other, a linear or branched C₁₋₆ hydrocarbon-based group bearing at least one primary, secondary or tertiary amine function or at least one quaternary nitrogen atom, and combinations thereof.

16. An aerosol device according to claim 14, wherein the hydrophobic block contains up to about 10 mol % of one or more water-soluble monomers selected from the group consisting of ethylenically unsaturated carboxylic acids, 2-acrylamido-2-methylpropanesulphonic acid, styrenesulphonic acid, vinylsulphonic acid, vinylphosphonic acid, acrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol, ethylene oxide, hydroxyethyl acrylate, hydroxypropyl acrylate, hydroxyethyl methacrylate, hydroxypropyl methacrylate, dimethyldiallylammonium chloride, methylvinylimidazolium chloride, 2-vinylpyridine, 4-vinylpyridine, 2-methyl-5-vinylpyridine, vinylamine, the monomers of formula H₂C═CR₁—CO—X₂ in which R₁ represents a hydrogen atom or a methyl group, X₂ represents a linear or branched C₁₋₆ hydrocarbon-based group bearing at least one primary, secondary or tertiary amine function or at least one quaternary nitrogen atom, or a group of formula NHR₂ or of formula NR₂R₃ in which R₂ and R₃ represent, independently of each other, a linear or branched C₁₋₆ hydrocarbon-based group bearing at least one primary, secondary or tertiary amine function or at least one quaternary nitrogen atom, and combinations thereof.

17. An aerosol device according to claim 14, wherein the hydrophobic block contains up to about 5 mol % of one or more water-soluble monomers selected from the group consisting of ethylenically unsaturated carboxylic acids, 2-acrylamido-2-methylpropanesulphonic acid, styrenesulphonic acid, vinylsulphonic acid, vinylphosphonic acid; acrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol, ethylene oxide, hydroxyethyl acrylate, hydroxypropyl acrylate, hydroxyethyl methacrylate, hydroxypropyl methacrylate, dimethyldiallylammonium chloride, methylvinylimidazolium chloride, 2-vinylpyridine, 4-vinylpyridine, 2-methyl-5-vinylpyridine, vinylamine, the monomers of formula H₂C═CR₁—CO—X₂ in which R₁ represents a hydrogen atom or a methyl group, X₂ represents a linear or branched C₁₋₆ hydrocarbon-based group bearing at least one primary, secondary or tertiary amine function or at least one quaternary nitrogen atom, or a group of formula NHR₂ or of formula NR₂R₃ in which R₂ and R₃ represent, independently of each other, a linear or branched C₁₋₆ hydrocarbon-based group bearing at least one primary, secondary or tertiary amine function or at least one quaternary nitrogen atom, and combinations thereof.

18. An aerosol device according to claim 1, wherein the block copolymer is water-soluble or water-dispersible.

19. An aerosol device according to claim 18, wherein the block copolymer is water-soluble.

20. An aerosol device according to claim 1, wherein the hydrophilic block represents at least about 60% to about 97% by weight of the linear block copolymer.

21. An aerosol device according to claim 1, wherein the styling composition includes from about 0.01% to about 10% by weight of the linear block copolymer.

22. An aerosol device according to claim 1, wherein the styling composition includes from about 0.1% to about 5% by weight of the linear block copolymer.

23. An aerosol device according to claim 1, wherein the styling composition has a water content of between about 30% and about 99.9% by weight of the composition.

24. An aerosol device according to claim 1, wherein the styling composition has a water content of between about 50% and about 99% by weight of the composition.

25. An aerosol device according to claim 1, wherein the styling composition has a water content of between about 75% and about 98% by weight of the composition.

26. An aerosol device according to claim 1, wherein the styling composition constitutes a hair lacquer.

27. A hair styling process comprising vaporizing the styling composition contained in the aerosol device of claim 1 onto wet or dry hair.

28. An aerosol device comprising:
a first compartment containing a styling composition containing a diblock copolymer including at least one hydrophilic block and at least one hydrophobic block, wherein said at least one hydrophilic block is poly(N-methyl-N-vinyl pyridinium iodide) and said at least one hydrophobic block is polystyrene, wherein said styling composition excludes a volatile organic compound, and
a second compartment containing a compressed gas, wherein said compressed gas is not a volatile organic compound.

29. The aerosol device of claim 28, wherein said styling composition in said first compartment does not contain any additive.
